(19) United States
(12) Patent Application Publication
(10) Pub. No.: US 2021/0406526 A1
(43) Pub. Date: Dec. 30, 2021

(54) INFORMATION PROCESSING DEVICE, INFORMATION PROCESSING METHOD, AND PROGRAM

(71) Applicant: Tamagawa Academy & University, Tokyo (JP)

(72) Inventors: Takashi OMORI, Tokyo (JP); Tetsuji YAMADA, Tokyo (JP)

(73) Assignee: Tamagawa Academy & University, Tokyo (JP)

(21) Appl. No.: 16/977,060

(22) PCT Filed: Mar. 1, 2019
(86) PCT No.: PCT/JP2019/008133

§ 371 Date: Aug. 31, 2020

(30) Foreign Application Priority Data
Mar. 1, 2018 (JP) 2018-036546

(51) Int. Cl. G06K 9/00; G06K 9/62

(52) U.S. Cl. CPC G06K 9/00335; G09B 19/00; G06K 9/00624; G06K 9/628

(57) ABSTRACT

The objective of the present invention is to appropriately estimate the psychological state or developmental characteristic of a subject receiving education or childcare. An observation result acquisition unit 113 extracts a behavior observation result for each of a plurality of persons for each of a plurality of units, one unit being defined a prescribed location during a prescribed time period. An object-of-interest estimation unit 114 estimates an object of interest of an individual on the basis of each obtained behavior observation result for each of the plurality of persons and each of the plurality of units. A field classification unit 115 classifies each of the plurality of units from among one or more types of fields formed by a group and one or more types of fields formed by a specific individual into one or more types of fields, on the basis of the estimation results from the object-of-interest estimation means for each of the plurality of persons and each of the plurality of units. When an object of interest for a prescribed person in a prescribed unit is estimated by the object-of-interest estimation means, a psychological trait estimation unit 116 estimates a psychological trait for a prescribed person on the basis of a difference between the average values for the behavior observation result for the prescribed person and the behavior observation result for other persons in the prescribed unit.

### TECHNICAL FIELD

The present invention relates to an information processing device, an information processing method, and a program.

### BACKGROUND ART

According to a technique conventionally suggested for a field of education or childcare, a problem content of difficulty or importance responsive to a learning level is offered to a subject receiving education or childcare (see patent document 1, for example).

Patent Document 1: Japanese Unexamined Patent Application, Publication No. 2015-102556

### DISCLOSURE OF THE INVENTION

### Problems to be Solved by the Invention

At present, however, in order to provide optimum education or childcare to a subject to receive the education or childcare, consideration is required to be given not only to a learning level but also to the psychological state or developmental characteristic of the subject. Conventional techniques including the one of patent document 1 do not meet such requirement satisfactorily.

The present invention has been made in view of the foregoing situation. An object of the present invention is to properly estimate the psychological state or developmental characteristic of a subject receiving education or childcare.

### Means for Solving the Problems

To fulfill the foregoing object, an information processing device according to one aspect of the present invention includes:

acquisition means, using a prescribed location in a prescribed time period as one unit, the acquisition means extracting a behavior observation result about a person on the basis of each of a plurality of the units for each of a plurality of persons;
object-of-interest estimation means, on the basis of the behavior observation result acquired for each of a plurality of the persons and each of a plurality of the units, the object-of-interest estimation means estimating an object of interest of an individual; and
field classification means, on the basis of a result of the estimation by the object-of-interest estimation means obtained for each of a plurality of the persons and each of a plurality of the units, the field classification means classifying each of a plurality of the units into one or more types of fields from among one or more types of fields formed by a group and one or more types of fields formed by a specific individual.

### Effects of the Invention

According to the present invention, the psychological state or developmental characteristic of a subject receiving education or childcare can be estimated properly.

### BRIEF DESCRIPTION OF THE DRAWINGS

FIG. 1 is a block diagram showing the configuration of an information processing system according to an embodiment of the present invention;

FIG. 2 is an explanatory view for explaining the outline of service according to the embodiment of the present invention;

FIG. 3 is a block diagram showing the hardware configuration of a server according to the embodiment of the present invention forming the information processing system in FIG. 1;

FIG. 4 is a functional block diagram showing exemplary functional configurations of the server in FIG. 3 and a client terminal;

FIG. 5 is a flowchart explaining a flow of a characteristic information estimation process performed by the server in FIG. 4;

FIG. 6 is an explanatory view for showing an example of an object of interest;

FIG. 7 is an explanatory view for showing an example of classification into a field;

FIG. 8 is an explanatory view for showing an example of classification into a field;

FIG. 9 is an explanatory view for showing an example of estimation of a psychological trait;

FIG. 10 is an explanatory view for showing an example of estimation of a psychological trait;

FIG. 11 is an explanatory view for showing an example of estimation of a developmental characteristic;

FIGS. 12-1 to 12-3 show a specific example of this service and shows an example in which a client (childcare worker) at an educational facility gives picture book reading to subjects (children);

FIGS. 13-1 to 13-3 show a specific example of this service and shows an example in which a target educational facility is in free time;

FIG. 14 shows an example of a technique of calculating a likelihood distance to become a basis for supporting the principles of interest estimation;

FIG. 15 is a table showing a percentage of correct answers of interest estimation; and

FIGS. 16-1 to 16-3 show an example of an analysis technique using a likelihood distance.

### PREFERRED MODE FOR CARRYING OUT THE INVENTION

An embodiment of the present invention will be described below using the drawings.

(Outline of Information Processing System)

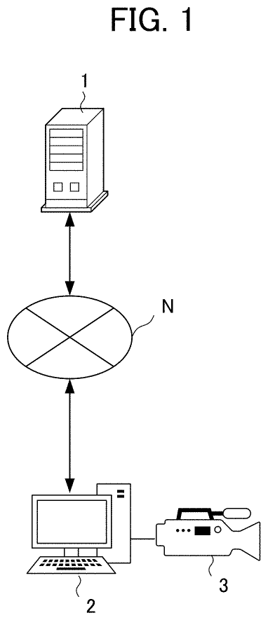

FIG. 1 shows the configuration of an information processing system according to the embodiment of the present invention.

The information processing system shown in FIG. 1 is used for providing service described below (hereinafter called “this service”). This service is to estimate the psychological trait or developmental characteristic of a person to be observed (hereinafter called “subject”). This service is usable in various types of regions (situations). In an example described in the embodiment, this service is used in childcare and educational fields. More specifically, this service is used at an educational facility, an education research institution, or an education administrative institution (hereinafter simply called an “educational facility”) to properly estimate the characteristic of a person receiving childcare or education (hereinafter called “subject”) such as a psychological trait or a developmental characteristic. In the following description, a person such as an administrator of the educational facility will be called “client.” The person such as an administrator of the educational facility includes an official responsible for management of a school, and additionally, an educator (teacher) to teach or educate the subject in a classroom, namely, a childcare worker.

The configuration of the information processing system in FIG. 1 includes a server 1, a client terminal 2, and an imaging device 3. The server 1 and the client terminal 2 are connected to each other via a prescribed network N such as the Internet. The server 1 is managed by a provider of this service. The client terminal 2 is managed by a client. The imaging device 3 is configured using a video camera, for example, and is located at the educational facility.

(Outline of this Service)

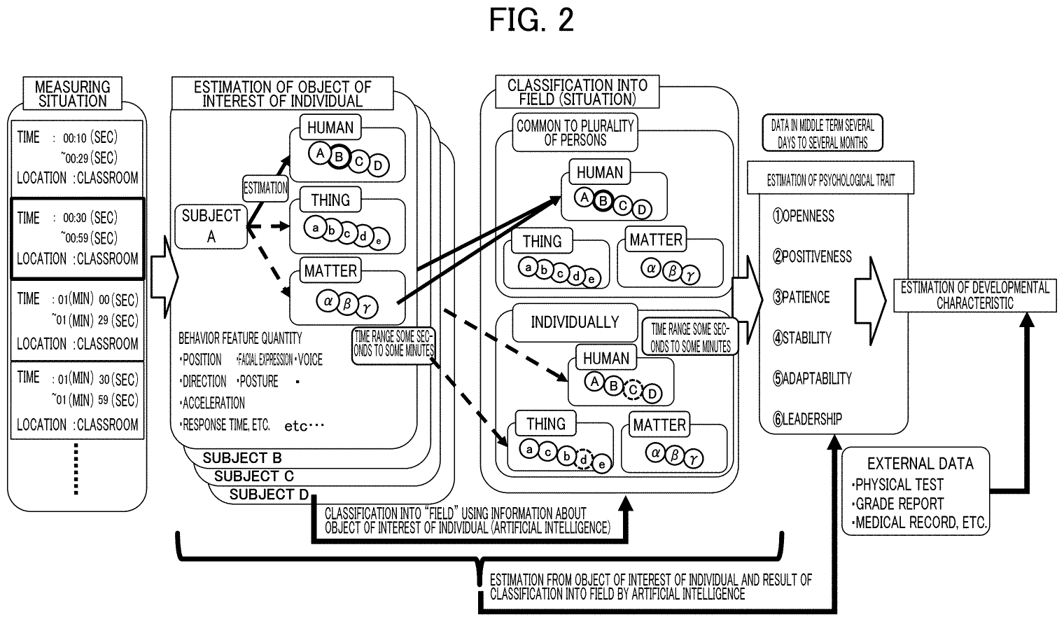

The outline of this service will be described next by referring to FIG. 2. FIG. 2 is an explanatory view for explaining the outline of this service according to the embodiment of the present invention. While not shown in the example of FIG. 2, there is a plurality of subjects such as students in a prescribed location at the educational facility that may be “classroom,” for example, and various types of behaviors of these subjects (subjects A to D, for example) are imaged by the imaging device 3 and observed. Namely, in the example of FIG. 2, the imaging device 3 is located in “classroom” and outputs data about captured images in a prescribed time period. The data about captured images is transmitted through the client terminal 2 to the server 1. The data about captured images includes various types of data about images captured by the imaging device 3, and various types of data about time when these captured images are obtained, for example. Namely, the server 1 acquires data about captured images using a prescribed location in a prescribed time period as one unit. One unit mentioned herein is shown in FIG. 2 as “measuring situation” in a plurality of partitioned rectangles. Such one unit will also be called “situation.” In the example of FIG. 2, a first situation is specified by a time period “00 (minute):10 (second) to 00 (minute):29 (second)” and a location “classroom.” Data about captured images in the first situation is acquired by the server 1. Likewise, a second situation is specified by a time period “00 (minute):30 (second) to 00 (minute):59 (second)” and a location “classroom.” Data about captured images in the second situation is acquired by the server 1. A third situation is specified by a time period “01 (minute):00 (second) to 01 (minute):29 (second)” and a location “classroom.” Data about captured images in the third situation is acquired by the server 1. A fourth situation is specified by a time period “01 (minute):30 (second) to 01 (minute):59 (second)” and a location “classroom.” Data about captured images in the fourth situation is acquired by the server 1.

Next, on the basis of the data about captured images in each “situation,” the server 1 acquires a behavior observation result about a subject in each “situation” for each of a plurality of the subjects A to D. Then, on the basis of the behavior observation result acquired for each of a plurality of the subjects A to D and each “situation,” the server 1 estimates an object of interest of an individual. An object of interest of an individual is estimated from one or more elements from among three elements including a human (a childcare worker in “classroom,” for example), a thing (a toy in the classroom, for example), and a matter (an event other than the human and the thing). Namely, an object of interest of an individual is estimated for the subject A for each of the first situation to the fourth situation. Likewise, an object of interest of an individual is estimated for the subject B for each of the first situation to the fourth situation. An object of interest of an individual is estimated for the subject C for each of the first situation to the fourth situation. An object of interest of an individual is estimated for the subject D for each of the first situation to the fourth situation.

As a specific example, the server 1 extracts a behavior feature quantity and environmental information about each of a plurality of the subjects A to D and each “situation” on the basis of corresponding data about captured images. Namely, the behavior observation result includes a behavior feature quantity and environmental information about at least a plurality of the subjects A to D. The behavior feature quantity mentioned herein is a feature quantity about the behavior of a subject. A quantity that can be obtained as the behavior feature quantity includes a position, a direction, a posture, an acceleration, a facial expression, voice (sound), and response time. The environmental information means information classified into three, “human,” “thing,” and “matter,” and information indicating external environment in which a subject in each “situation” is placed. Namely, a behavior feature quantity and environmental information are extracted for the subject A in each of the first situation to the fourth situation. Likewise, a behavior feature quantity and environmental information are extracted for the subject B in each of the first situation to the fourth situation. A behavior feature quantity and environmental information are extracted for the subject C in each of the first situation to the fourth situation. A behavior feature quantity and environmental information are extracted for the subject D in each of the first situation to the fourth situation.

Then, on the basis of the behavior feature quantity and the environmental information, the server 1 estimates an object of interest of an individual for each of a plurality of the subjects A to D and each “situation.” As a specific example, a childcare worker (“human”) and a toy (“thing”) are extracted as the environmental information for the subject A in the first situation. If a posture included in the behavior feature quantity about the subject A is pointed toward the childcare worker, an object of interest of an individual is estimated to be the childcare worker (“human”).

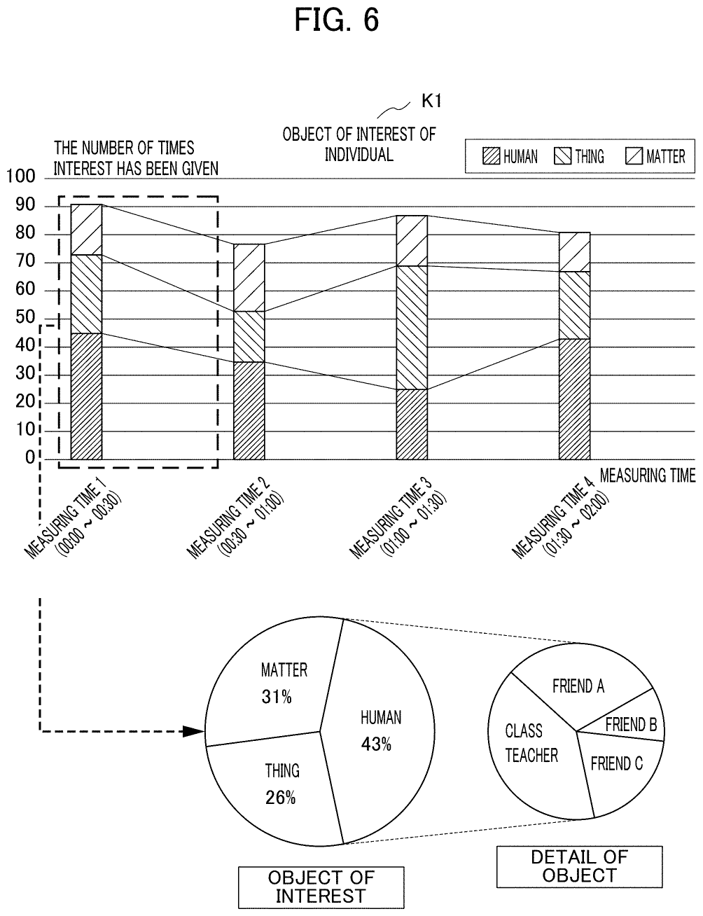

Any period and any location are applicable to a situation (one unit). Regarding a period, however, setting a short period (from some seconds to some hours) allows more detailed estimation of an object of interest. Estimation of an object of interest of each of a plurality of subjects will be described later in detail by referring to FIG. 6.

Next, on the basis of a result of “estimation of an object of interest of an individual” for each of a plurality of the subjects A to D and each “situation,” the server 1 classifies each “situation” into one or more types of “fields” from among one or more types of “fields” formed by a group and one or more types of “fields” formed by a specific individual. As a specific example, in the embodiment, each “situation” is classified into one or more types from among six types (first classification to sixth classification) of “fields” using a frequency of occurrence and a distribution of an object of interest of an individual estimated for each of a plurality of the subjects A to D.

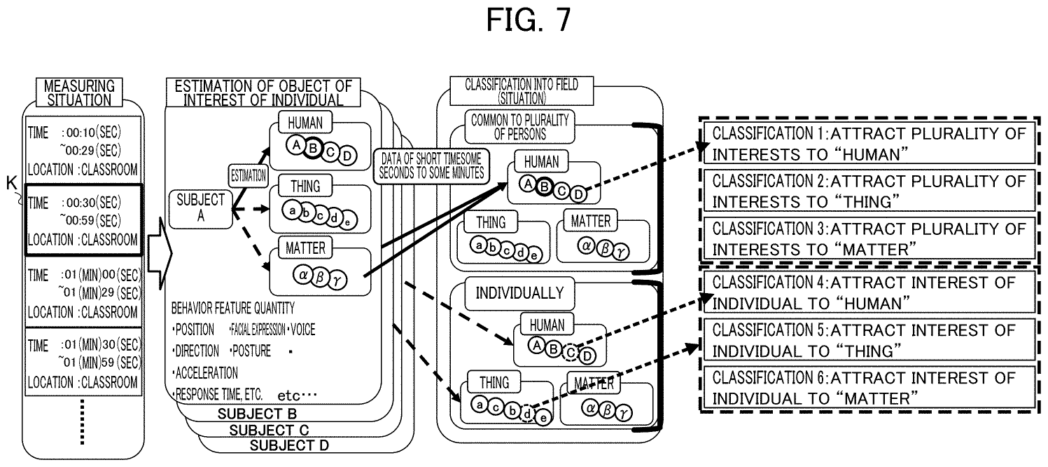

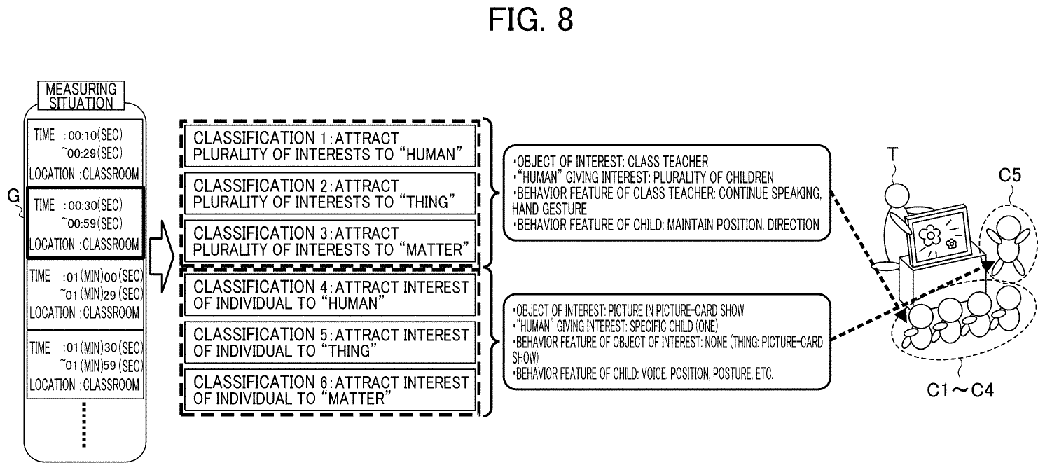

“Fields” in the first classification to the third classification are “field” types formed by a group.
“Field” in the first classification is a type of attracting interests of a plurality of subjects to “human.” “Field” in the second classification is a type of attracting interests of a plurality of subjects to “thing.” “Field” in the third classification is a type of attracting interests of a plurality of subjects to “matter.” “Fields” in the fourth classification to the sixth classification are “field” types formed by a specific individual.
“Field” in the fourth classification is a type of attracting an interest of a specific individual (specific subject) to “human.” “Field” in the fifth classification is a type of attracting an interest of a specific individual (specific subject) to “thing.” “Field” in the sixth classification is a type of attracting an interest of a specific individual (specific subject) to “matter.” For example, if objects of interest of the three subjects A, B, and C from among the subjects A to D are estimated to be a childcare worker (“human”) in the first situation, the first situation is classified into “field” in the first classification. If an object of interest of the subject D is an image (“thing”) in the first situation, the first situation is also classified into “field” in the fifth classification. Namely, “situation” can be classified not only into one “field” but also into a plurality of “fields.” In this way, the server 1 allows estimation of a wide range of objects of interest including that of an individual and those of a large group, and allows classification of a prescribed “situation” into a single or a plurality of “fields.” Such a series of processes of classifying each prescribed “situation” into a single or a plurality of “fields (first classification to sixth classification)” will collectively be called “classification into field.” This classification of “situation” can be made using various types of techniques relating to machine learning such as deep learning and deep structured learning. The classification of “situation” will be described later in detail by referring to FIGS. 7 and 8.

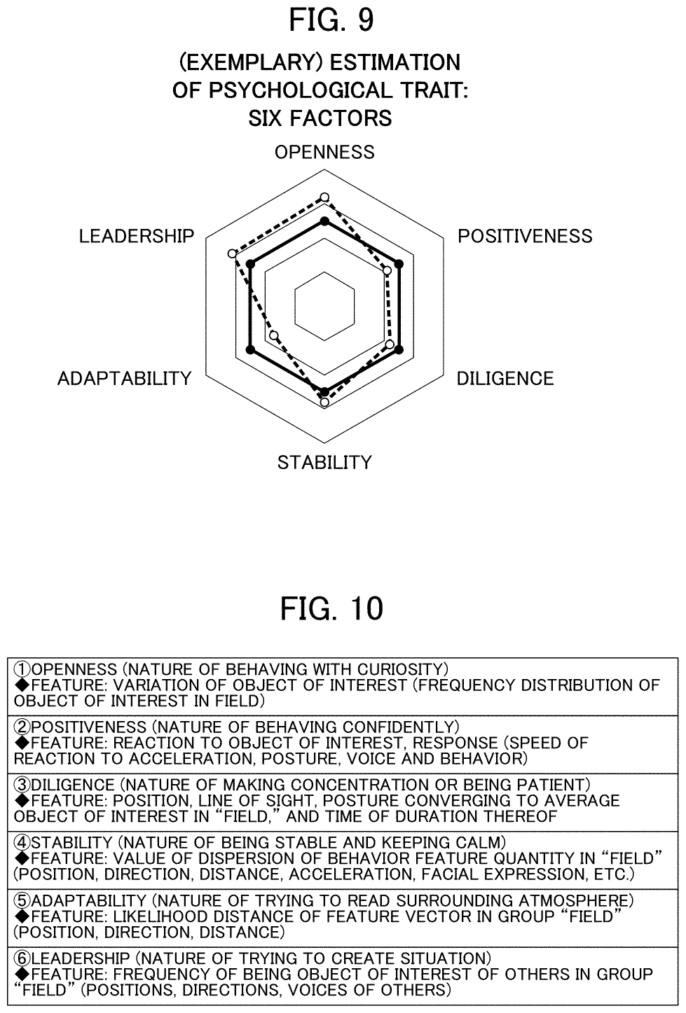

Next, on the basis of a result of “classification into field” for each of a plurality of the subjects A to D and each “situation,” the server 1 estimates the psychological trait of each subject. As a specific example, in the embodiment, the psychological trait of each of a plurality of the subjects A to D is stochastically estimated by analyzing an object of interest of an individual estimated for each of a plurality of the subjects A to D, and deviation between a behavior feature quantity of an individual for each of a plurality of the subjects A to D at the time of the estimation and the behavior feature quantity in a corresponding “field” from an average. To be more specific, a psychological trait is estimated by analyzing deviation between a behavior feature quantity of an individual for each of a plurality of the subjects A to D and the behavior feature quantity in a corresponding “field” from an average. For this reason, a psychological trait is desirably estimated using information accumulated in a medium term of from several days to several months, for example. Using such accumulated data is expected to improve estimation accuracy. Estimation of a psychological trait includes estimation of developmental disability, bullying, and stress resistance, for example. Such estimation of a psychological trait can also be made using various types of techniques relating to machine learning such as deep learning and deep structured learning. Estimation of a psychological trait and a developmental characteristic will be described later in detail by referring to FIGS. 9 to 11.

The outline of this service is as has been described above. The following describes the hardware configuration and functional block diagram of the information processing system of the embodiment for realizing this service described above.

### (Hardware Configuration of Information Processing System)

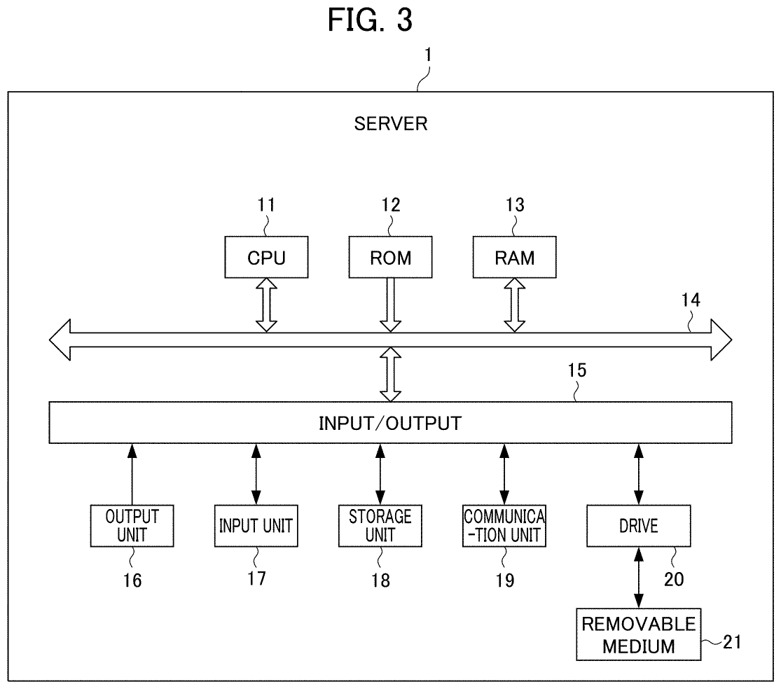

FIG. 3 is a block diagram showing the hardware configuration of the server 1 forming the information processing system in FIG. 1.

The server 1 includes a central processing unit (CPU) 11, a read only memory (ROM) 12, a random access memory (RAM) 13, a bus 14, an input/output interface 15, an output unit 16, an input unit 17, a storage unit 18, a communication unit 19, and a drive 20.

The CPU 11 performs various types of processes by following a program stored in the ROM 12 or a program loaded from the storage unit 18 onto the RAM 13. If appropriate, the RAM 13 stores data necessary for implementation of the various processes by the CPU 11, or the like.

The CPU 11, the ROM 12, and the RAM 13 are connected to each other via the bus 14. The input/output interface 15 is further connected to the bus 14. The output unit 16, the input unit 17, the storage unit 18, the communication unit 19, and the drive 20 are connected to the input/output interface 15.

The output unit 16 is configured using any type of liquid crystal display, for example, and is used for outputting various types of information. The input unit 17 is configured using any type of hardware, for example, and is used for inputting various types of information. The storage unit 18 is configured using a hard disk or a dynamic random access memory (DRAM), for example, and is used for storing various types of data. The communication unit 19 controls communication with a different device (in the example of FIG. 1, client terminal 2) via the network N including the Internet.

The drive 20 is prepared according to demand. If appropriate, a removable medium 21 configured using a magnetic disk, an optical disk, a magneto-optical disk, or a semiconductor memory, for example, is fitted to the drive 20. A program read from the removable medium 21 using the drive 20 is installed on the storage unit 18 according to demand. The removable medium 21 can also be used for storing various types of data stored in the storage unit 18 in the same way as the storage unit 18.

The client terminal 2 include structures basically the same as those of the server 1, so that these structures will be omitted from this description.

Various types of hardware and various types of software in the server 1 and the client terminal 2 in FIG. 3 described above work cooperatively to perform a series of processes, thereby allowing provision of this service.

More specifically, the client terminal 2 transmits data about captured images taken by a client to the server 1. Timing of transmitting the data about captured images to the server 1 may be determined arbitrarily. The data may be transmitted automatically with prescribed timing (at daily intervals, for example).

When the data about captured images transmitted from the client terminal 2 is acquired, the server 1 analyzes the acquired data about captured images. Then, on the basis of the data about captured images, the server 1 estimates an object of interest of each subject for each of a plurality of “situations,” classifies the “situation” into the six types of “fields,” and estimates the psychological trait or developmental characteristic of each subject, as described above. Then, the server 1 transmits these various types of estimated information to the client terminal 2.

When the various types of estimated information are received from the server 1, the client terminal 2 presents the received information to the client.

(Functional Block Diagram of Information Processing System)

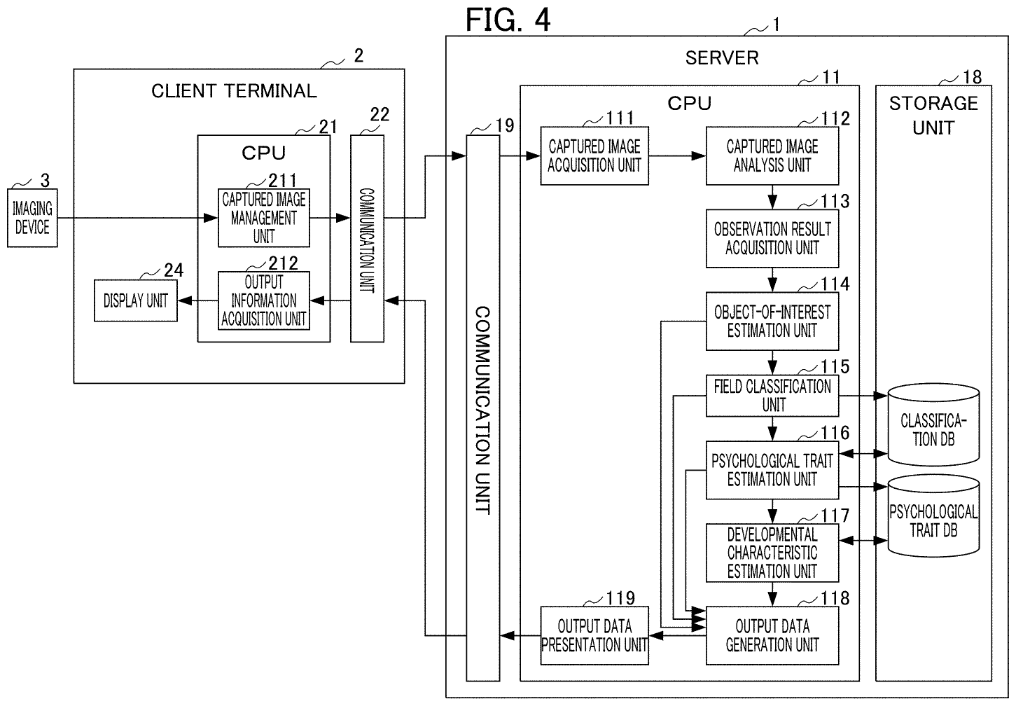

To realize the series of processes described above, the information processing system including the server 1 and the client terminal 2 has a functional configuration such as that shown in FIG. 4. FIG. 4 is a functional block diagram showing exemplary functional configurations of the server 1, the client terminal 2, and the imaging device 3.

In a CPU 21 of the client terminal 2, a captured image management unit 211 and an output information acquisition unit 212 become functional.

The captured image management unit 211 of the client terminal 2 executes control for transmitting data about captured images taken by the imaging device 3 to the server 1 through a communication unit 22. The data about captured images taken by the imaging device 3 is transmitted from the imaging device 3 to the client terminal 2 by a wire or wireless communication system.

The output information acquisition unit 212 of the client terminal 2 acquires various types of information (hereinafter called “output information”) about the psychological trait or developmental characteristic of a subject transmitted from the server 1 through the communication unit 22. The output information acquisition unit 212 executes control for displaying the acquired output information on a display unit 24.

In the CPU 11 of the server 1, a captured image acquisition unit 111, a captured image analysis unit 112, an observation result acquisition unit 113, an object-of-interest estimation unit 114, a field classification unit 115, a psychological trait estimation unit 116, a developmental characteristic estimation unit 117, an output data generation unit 118, and an output data presentation unit 119 become functional.

Using a prescribed location in a prescribed time period as one unit, the captured image acquisition unit 111 of the server 1 acquires data about captured images through the communication unit 19 transmitted from the client terminal 2.

The captured image analysis unit 112 of the server 1 analyzes the data about captured images acquired by the captured image acquisition unit 111. More specifically, on the basis of the data about captured images acquired by the captured image acquisition unit 111, the captured image analysis unit 112 links the acquired image data with “situation (first situation, for example).”

Using a prescribed location in a prescribed time period as one unit, the observation result acquisition unit 113 of the server 1 extracts a behavior observation result about a person on the basis of each of a plurality of the units for each of a plurality of persons. More specifically, on the basis of the data about captured images in each “situation (first situation to fourth situation, for example),” the observation result acquisition unit 113 extracts a behavior observation result about a subject in each “situation” for each of a plurality of the subjects A to D.

On the basis of the behavior observation result acquired for each of a plurality of the persons and each of a plurality of the units, the object-of-interest estimation unit 114 of the server 1 estimates an object of interest of an individual. More specifically, on the basis of a behavior feature quantity and environmental information extracted by the observation result acquisition unit 113 for each of a plurality of the subjects A to D and each “situation (first situation to fourth situation, for example),” the object-of-interest estimation unit 114 estimates an object of interest of an individual for each of a plurality of subjects. A method of estimating an object of interest will be described later in detail using FIG. 6, and the like.

On the basis of a result of the estimation by the object-of-interest estimation means obtained for each of a plurality of the persons and each of a plurality of the units, the field classification unit 115 of the server 1 classifies each of a plurality of the units into one or more types of fields from among one or more types of fields formed by a group and one or more types of fields formed by a specific individual. Specifically, on the basis of a result of “estimation of an object of interest of an individual” for each of a plurality of subjects (subjects A to D, for example) and each “situation (first situation to fourth situation, for example)” estimated by the object-of-interest estimation unit 114, the field classification unit 115 classifies each “situation” into one or more types of “fields” from among one or more types of fields formed by a group and one or more types of fields formed by a specific individual. More specifically, the field classification unit 115 classifies each “situation” into one or more types from among the six types (first classification to sixth classification) of “fields” using a frequency of occurrence and a distribution of an object of interest of an individual estimated for each of a plurality of subjects. Further, the field classification unit 115 stores a result of the “classification into field” into a classification DB 500. A method of the “classification into field” will be described later in detail using FIG. 8, and the like.

When the object-of-interest estimation means estimates an object of interest of a prescribed person in a prescribed unit, the psychological trait estimation unit 116 of the server 1 estimates the psychological trait of the prescribed person on the basis of a difference between a behavior observation result about the prescribed person and an average of behavior observation results about other persons in the prescribed unit. More specifically, the psychological trait estimation unit 116 estimates the psychological trait of each subject on the basis of “result of classification into field” for each of a plurality of the subjects A to D and each “situation.” As a specific example, in the embodiment, the psychological trait of each of a plurality of the subjects A to D is stochastically estimated by analyzing an object of interest of an individual estimated for each of a plurality of the subjects A to D, and deviation between a behavior feature quantity of an individual for each of a plurality of the subjects A to D at the time of the estimation and the behavior feature quantity in a corresponding “field” from an average. The psychological trait estimation unit 116 stores a result of the estimation of the psychological trait estimated for each of a plurality of the subjects A to D into a psychological trait DB 600. The psychological trait estimated by the psychological trait estimation unit 116 mentioned herein includes six natures of “openness,” “positiveness,” “diligence,” “stability,” “adaptability,” and “leadership,” for example. A method of estimating such a psychological trait will be described later in detail using FIG. 9, and the like.

On the basis of a difference between a feature of a long-term transition of the psychological trait of the prescribed person estimated by the psychological trait estimation means and a feature of a long-term transition of a standard psychological trait, the developmental characteristic estimation unit 117 of the server 1 estimates the developmental characteristic of the prescribed person. As a specific example, in the embodiment, the developmental characteristic estimation unit 117 estimates the developmental characteristic of each of a plurality of the subjects A to D on the basis of a difference between the tendency of a long-term transition of the psychological trait of each subject estimated for each of a plurality of the subjects A to D and the tendency of a long-term transition of an average psychological trait in a corresponding “field.”

On the basis of a result of the estimation of an object of interest of each subject estimated by the object-of-interest estimation unit 114, a result of “classification into field” classified by the field classification unit 115, a result of the estimation of the psychological trait of each subject estimated by the psychological trait estimation unit 116, a result of the estimation of the developmental characteristic of each subject estimated by the developmental characteristic estimation unit 117, and others, the output data generation unit 118 of the server 1 generates output data. The output data generation unit 118 stores information in the generated output data into a database not shown provided in the storage unit 18.

The output data presentation unit 119 of the server 1 executes control of transmitting the output data generated by the output data generation unit 118 to the client terminal 2 through the communication unit 19.

### (Characteristic Information Estimation Process)

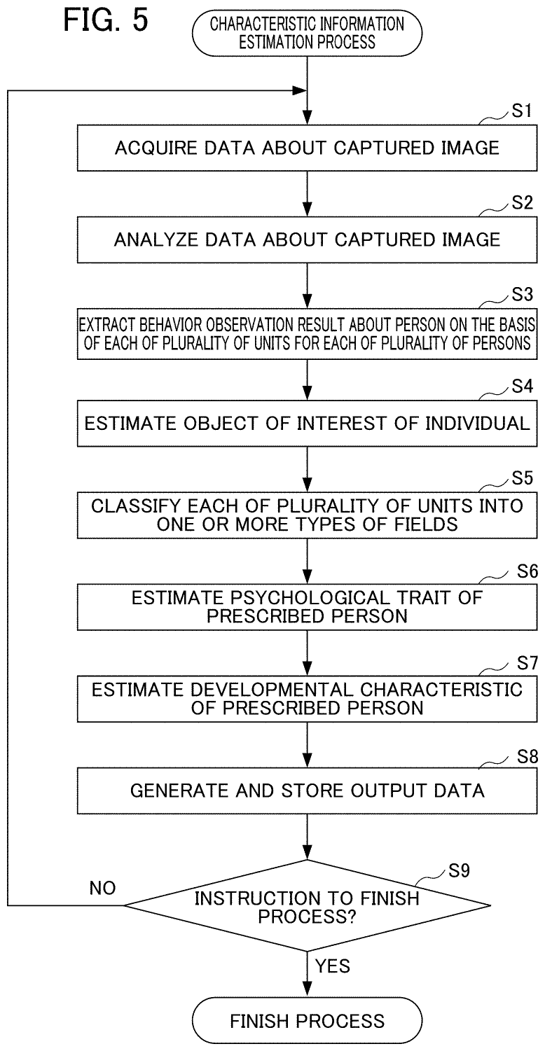

A characteristic information estimation process performed by the server 1 having the functional configuration in FIG. 4 will be described next by referring to FIG. 5. FIG. 5 is a flowchart explaining a flow of the characteristic information estimation process performed by the server 1.

In step S1, using a prescribed location in a prescribed time period as one unit, the captured image acquisition unit 111 acquires data about captured images through the communication unit 19 transmitted from the client terminal 2.

In step S2, the data about captured images acquired in step S1 is analyzed. More specifically, on the basis of the data about captured images acquired by the captured image acquisition unit 111, the captured image analysis unit 112 links the acquired image data with “situation (first situation, for example).”

In step S3, using a prescribed location in a prescribed time period as one unit, the observation result acquisition unit 113 extracts a behavior observation result about a person on the basis of each of a plurality of the units for each of a plurality of persons. More specifically, on the basis of the data about captured images in each “situation (first situation to fourth situation, for example),” the observation result acquisition unit 113 extracts a behavior observation result about a subject in each “situation” for each of a plurality of the subjects A to D.

In step S4, on the basis of the behavior observation result acquired for each of a plurality of the persons and each of a plurality of the units, the object-of-interest estimation unit 114 estimates an object of interest of an individual. More specifically, on the basis of a behavior feature quantity and environmental information extracted in step S3 for each of a plurality of the subjects A to D and each “situation (first situation to fourth situation, for example),” the object-of-interest estimation unit 114 estimates an object of interest of an individual for each of a plurality of subjects.

### (Estimation of Object of Interest)

The foregoing estimation of an object of interest in step S4 will be described in detail by referring to FIG. 6. FIG. 6 is a view for explaining an example of an object of interest.

FIG. 6 shows an example of estimation of an object of interest of each subject in a scene (childcare scene) in which children make activities in a kindergarten, for example. The example of FIG. 6 shows that an object of interest of one child (subject) in a situation K1 (measuring time 1, classroom) is occupied by “43%” of human, “26%” of thing, and “31%” of matter. The example also shows that the object of interest specifically includes “fried A,” “friend B,” “friend C,” and “class teacher.” As a specific example, an object of interest is estimated in this case from the “position,” “direction,” “distance,” “time (time when an object involved is being watched, for example)” etc. of a subject relative to “human,” “thing,” and “matter” identified using environmental information. As a more specific example, in the presence of familiarity with a childcare worker in charge or in the presence of a close friend, a distance of a subject to the childcare worker or the fried is short and a line of sight is directed toward the childcare worker or the friend. Thus, the childcare worker or the friend can be estimated to be an object of interest from the posture of the subject accompanied by a head or shoulders, or a length of time when the line of sight is maintained. The subject is to stay longer near the object of interest. In summary, an object of interest is estimated from a behavior feature quantity determined when the subject pays attention to an involved object intentionally. Such estimation of an object of interest can be realized by acquiring information in a short term such as from some seconds to some hours.

The description continues by referring back to FIG. 5. In step S5, on the basis of a result of the estimation by the object-of-interest estimation means obtained for each of a plurality of the persons and each of a plurality of the units, the field classification unit 115 classifies each of a plurality of the units into one or more types of fields from among one or more types of fields formed by a group and one or more types of fields formed by a specific individual. More specifically, on the basis of a result of “estimation of an object of interest of an individual” estimated in step S4 for each of a plurality of subjects (subjects A to D, for example) and each “situation (first situation to fourth situation, for example),” the field classification unit 115 classifies each “situation” into one or more types of “fields” from among one or more types of fields formed by a group and one or more types of fields formed by a specific individual.

(Classification into “Field”)

A method of the foregoing “classification into field” in step S4 will be described in detail by referring to FIGS. 7 and 8, and the like. FIGS. 7 and 8 are explanatory views for explaining an example of a method of “classification into field.”

FIG. 7 shows an example of actually classifying a prescribed “situation K (time: 0030 (second) to 0059 (second), location: classroom)” into a “field” type (first classification to sixth classification). The classification into a “field” type mentioned herein is made using a behavior feature (position or direction, for example) and environmental information (arrangement of an object or a person, for example) obtained simultaneously with estimation of an object of interest of each subject. In this classification into a “field” type, it can be judged through object recognition, for example, whether classification is to be classified into a “field” type (first classification to third classification) of a group to which objects of interest of a plurality of persons converge, or into a “field” type (fourth classification to sixth classification) of an individual.

Next, by referring to FIG. 8, a specific method of “classification into field” will be described in detail using a scene in which a picture-card show is presented at a kindergarten (childcare scene).

Regarding “classification into field,” discrimination is first made between a “field” of an interest formed by a group (first classification to third classification) and a “field” of an interest formed by an individual (fourth classification to sixth classification). In the example of FIG. 8, for example, a childcare worker T presents a picture-card show, and thus the interests of many subjects (children C1 to C4) are estimated to be directed to the childcare worker T. More specifically, the children C1 to C4 exhibit behavior features such as sitting down at positions where the picture-card show is easy to see, for example, with lines of sight directed toward the childcare worker T. The objects of interest of the children C1 to C4 are estimated to be “human” (childcare worker T). In this case, a “situation G (time: 0030 (second) to 0059 (second), location: classroom)” in which the children Cl to C4 participate is classified into the first classification. On the other hand, it is assumed that, even in the same situation G, there is a child C5 having an interest pointed toward an elephant in the picture-card show, standing and walking, and shouting “It's elephant!” in a loud voice. In this case, as shown in FIG. 8, the child C5 is in a posture of standing upright and pointed toward the picture-card show to get closer to an object of interest (elephant in the picture-card show). It can be determined from these behavior feature quantities that, in this “situation,” an object of interest of the child C5 (one subject) is “thing” (elephant in the picture-card show). Thus, the “situation G (time: 0030 (second) to 0059 (second), location: classroom)” in which the child C5 participates is classified into the fifth classification. As described above, even if the children C1 to C5 participate in the same “situation G (time: 0030 (second) to 0059 (second), location: classroom),” this situation can be classified into “fields” of a plurality of classifications (first classification and fifth classification, for example). This “situation” is determined by prescribed time (time unit) and a prescribed location. Thus, even in the same scene (childcare scene), classification of “situation” is still subject to change with the passage of time. In this way, this service allows estimation of an object of interest including that of an individual and those of a large group in each of a plurality of “situations,” and allows classification of “situation” (“classification into field”). As described above, this classification into “field” can be made using various types of techniques relating to machine learning such as deep learning and deep structured learning.

The description continues by referring back to FIG. 5. In step S6, when the object-of-interest estimation means estimates an object of interest of a prescribed person in a prescribed unit, the psychological trait estimation unit 116 estimates the psychological trait of the prescribed person on the basis of a difference between a behavior observation result about the prescribed person and an average of behavior observation results about other persons in the prescribed unit. More specifically, the psychological trait estimation unit 116 estimates the psychological trait of each subject on the basis of “result of classification into field” about each of a plurality of the subjects A to D and each “situation.”

### (Estimation of Psychological Trait)

The foregoing estimation of an object of interest in step S6 will be described in detail by referring to FIGS. 9 and 10. FIGS. 9 and 10 are views for explaining an example of estimation of a psychological trait. Referring to FIGS. 9 and 10, a psychological trait is composed of six factors (openness, positiveness, diligence, stability, adaptability, and leadership), for example.

The openness is one factor of a psychological trait and is a nature of behaving with curiosity. For example, if various types of “humans,” “things,” and “matters” are widely estimated to be objects of interest from a nature of being aware of anything quickly and going to see anything, this nature may be judged to be the openness. Such broadness of objects of interest is estimated from a difference between a relationship of uniformity/concentration of a frequency distribution of an object of interest of each subject and an average of objects of interest in each “field.” Namely, a feature of the openness lies in great variation (broadness) of objects of interest (frequency distribution of objects of interest in each “field”). The presence of too much variation is considered to exhibit a tendency toward distraction, so that greater variation does not always bring a more favorable result.

The positiveness is one factor of a psychological trait and is a nature of behaving confidently. For example, in a case where positive behaviors are observed such as being responsive to a question quickly, imitating the motion of a childcare worker immediately, lining up quickly, and going to a locker readily in the morning for preparation, this case may be judged to be the positiveness. Such positiveness is estimated from a speed or a posture of going toward an object of interest, for example. As an example, the presence of the positiveness is determined if a speed of going toward an object of interest increases, posture leans a forward and subject is chest-out and a step has large strides. If the presence of the positiveness is estimated, time of reaction is shortened in response to an approach from an object of interest (inquiry: imitation of a motion or a question). Such a speed or posture of going toward the object of interest (response) is estimated from a difference between a response from a subject and an average of responses in each “field.” Namely, a feature of the positiveness lies in quick reaction and a quick response (speed of reaction to acceleration, posture, voice, and behavior) to an object of interest.

The diligence is one factor of a psychological trait and is a nature of making concentration or being patient. More specifically, the presence of the diligence is estimated from time of duration of watching a picture-card show, drawing a picture in picture drawing time, listening to what a childcare worker is saying, sitting down and waiting, or studying at a desk, for example. In the presence of the diligence, as a result of making concentration on an object of interest or being patient, the direction of a head or a body relative to the object of interest or a position relative to the object of interest are maintained for certain time. This duration time of maintaining interest in the object of interest is estimated from a difference between a duration time of each subject and an average of duration times in “situation.” Namely, a feature of the diligence lies in long duration time for a position, a line of sight, and a posture converging to an average object of interest in a “field.”

The stability is one factor of a psychological trait and is a nature of being stable and keeping calm. For example, the presence of the stability is judged on the basis of criteria such as participating in an activity with a smile, crying, fighting, clasping a hand or cloth of a childcare worker continuously, starting to walk around suddenly, shouting, or the like. The stability becomes lower as a difference (deviation) of a behavior of a subject in each “field” from an average in each “field” becomes greater. On the other hand, if a behavior of a subject in each “field” is close to an average in each “field” and dispersion is small, the subject can be said to have high stability. Namely, a feature of the stability lies in small dispersion (outlier) of a behavior feature quantity (position, direction, distance, acceleration, facial expression, or the like) in a “field.” If the stability is low as a result of a high level of anxiety, for example, a subject exhibits an attachment behavior to a specific “human” or “thing.” If a subject is not good at waiting, the subject may walk around or make a fight. This generates a momentum (acceleration) or voice (loud voice) toward a specific “human” to increase dispersion of a behavior feature quantity. Conversely, if the stability is high, a behavior feature quantity is close to an average and dispersion decreases. Such dispersion of a behavior feature quantity is estimated from a difference (deviation) from an average of behavior feature quantities of all subjects in a “field (classified field)” in which each subject participates.

The adaptability is one factor of a psychological trait and is a nature of trying to read the surrounding atmosphere (imagining a situation from the atmosphere). In a case where an observed behavior is for imagining a situation from the atmosphere such as watching a picture-card show or a picture book, having a class in a group, waiting in a line, or starting fixing up, this case may be judged to be the adaptability. This adaptability is estimated in a “field” of a group and from the closeness of a likelihood distance from a main feature (an average of postures or directions, for example, in a “field” of reading a picture-card show). Namely, a feature of the adaptability lies in that a likelihood distance (position, direction, distance) is short between a feature vector of an individual and an average of feature vectors of a group in a “field.” While the adaptability has a characteristic similar to that of the stability, it is used mainly for estimating ease of adaptation to a “field” of a group (society).

The leadership is one factor of a psychological trait and is a nature of trying to create a situation. For example, if a behavior of organizing a group such as deciding a relay team or receiving a request for help from a child in trouble (receiving a question about an incomprehensive issue) is observed, this behavior may be judged to be the leadership. This leadership is estimated from a frequency of being an object of interest of other persons in a “field” of a group. Namely, a subject having the leadership talks to the group or puts forward a suggestion to be relied on by the others, thereby becomes an object of interest of the others at a high frequency (that is, there is a strong tendency of behavior feature quantities of the others such as positions or directions of being pointed toward the subject). Namely, a feature of the leadership lies in a high frequency of becoming an object of interest of other persons (positions, directions, voices of the others) in a “field” of a group. The psychological trait estimation unit 116 stores data about the estimated psychological trait of each subject (hereinafter called “psychological trait data”) into the psychological trait DB 600.

As described above, the six factors are estimated as a psychological trait. This estimation is particularly desirably made on the basis of data analysis in a medium term of from several days to several months. Using accumulation of such data about psychological trait estimation is expected to improve estimation accuracy. As described above, various types of techniques relating to machine learning such as deep learning and deep structured learning are available for use in the server 1, and this is expected to improve estimation accuracy further.

The description continues by referring back to FIG. 5. In step S7, on the basis of a difference between a feature of a long-term transition of the psychological trait of the prescribed person estimated by the psychological trait estimation means and a feature of a long-term transition of a standard psychological trait, the developmental characteristic estimation unit 117 estimates the developmental characteristic of the prescribed person. As a specific example, in the embodiment, the developmental characteristic estimation unit 117 estimates the developmental characteristic of each of a plurality of the subjects A to D on the basis of a difference between the tendency of a long-term transition of the psychological trait of the prescribed person estimated for each of a plurality of the subjects A to D and the tendency of a long-term transition of an average psychological trait in a corresponding “field.”

### (Estimation of Developmental Characteristic)

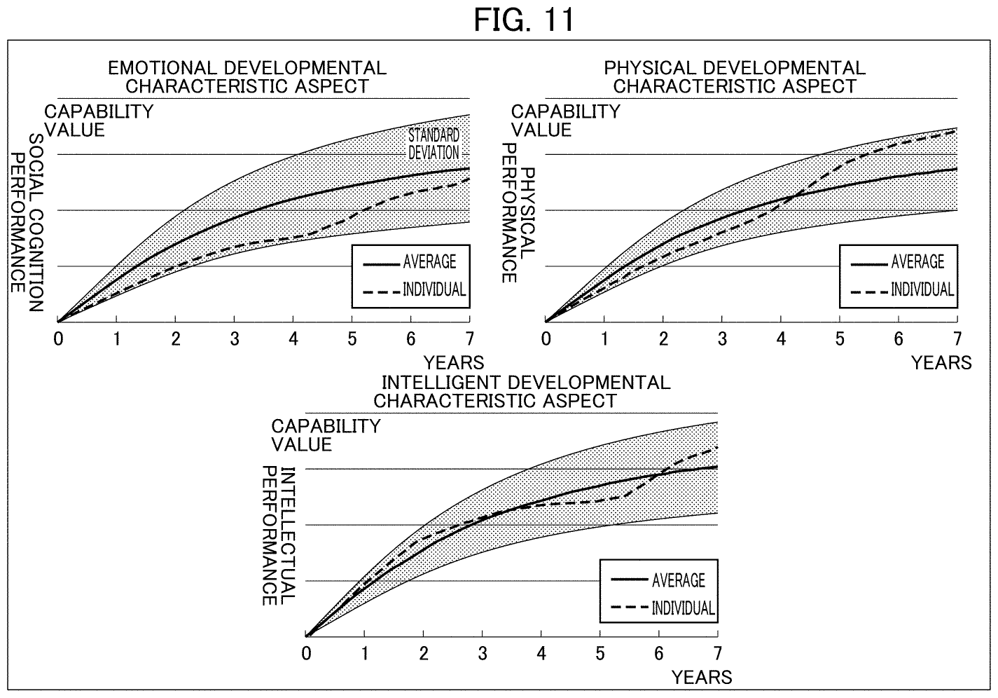

The foregoing estimation of a developmental characteristic in step S7 will be described by referring to FIG. 11. FIG. 11 is a view for explaining an example of estimation of a developmental characteristic.

As shown in FIG. 11, a developmental characteristic is classified into three aspects (emotional developmental characteristic aspect, physical developmental characteristic aspect, and intelligent developmental characteristic aspect). Estimation of a developmental characteristic will be described, particularly by referring to FIG. 11. For estimation of a developmental characteristic, it is particularly preferable to construct a standard developmental model using big data about psychological traits accumulated by a long-term measurement for several years. Such collection of massive data may be used as a basis for constructing a standard developmental model by overall means, and deviation of an individual (subject) from the standard developmental model may be estimated. As a specific example, the example of FIG. 11 is formed by simulating the developmental characteristic of a child for seven years from a birth (from entry into a nursery school to entry into an elementary school), and this example shows three aspects including “emotional developmental characteristic aspect,” “physical developmental aspect,” and “intelligent developmental aspect.” In other words, this example shows “emotional” growth, “physical” growth, and “intelligent” growth in a visualized manner. Namely, unlike a conventional developmental index, a developmental characteristic estimated by the developmental characteristic estimation unit 117 is expected to be used for estimating the characteristic of individual character development through long-term measurement of psychological traits. In this example, the emotional developmental characteristic aspect is defined as an aspect expressing performance relating to humanity (personality) such as non-cognitive performance or social emotional skill. Such a developmental characteristic is expected to be applied to an intelligence test or a physical test, for example. In addition to an intelligence test or a physical test, such a developmental characteristic is also expected to be used for deriving a comprehensive developmental index of a human, and the like through synchronization with an existing medical record, a grade report, or external data such as SNS information, for example. A subject of estimation of a developmental characteristic is not always limited to a child but the estimation is also applicable to a case of outputting change in transition of the developmental characteristic of an individual in a considerably long term such as from an adulthood to an old age.

The description continues by referring back to FIG. 5. In step S8, on the basis of a result of the estimation of an object of interest of each subject estimated in step S4, a result of the “classification into field” classified in step S5, a result of the estimation of the psychological trait of each subject estimated in step S6, a result of the estimation of the developmental characteristic of each subject estimated in step S7, and others, the output data generation unit 118 generates output data. The output data generation unit 118 stores information in the generated output data into the database not shown provided in the storage unit 18.

In step S9, the server 1 judges whether instruction to finish the process has been received. While the instruction to finish the process is not particularly limited, this instruction of the embodiment employs so-called cut-off instruction for a power supply of the server 1, for example. Namely, as long as the instruction to cut off the power supply of the server 1 is not given, a judgment NO is judged in step S9. Then, the process returns to step S1 and the subsequent steps are repeated. By contrast, if instruction for state change such as change to a sleep state is given to the server 1, a judgment YES is judged in step S9. Then, the characteristic information estimation process performed by the server 1 is finished.

While the embodiment of the present invention has been described above, the present invention is not limited to the foregoing embodiment. The effects described in the embodiment are merely a list of the most preferable effects resulting from the present invention. Effects achieved by the present invention should not be limited to those described in the embodiment.

While not shown, in this service, an annotation process is not always required to be performed manually but it may be performed semi-automatically. As a specific example, according to an applicable specification, information about a subject including an attribute such as a name or a sex, captured images, tracking data, and a behavior observation result is added semi-automatically, and a dialog is displayed in which a behavior feature quantity is editable. As another example, this dialog may be provided with an input form for allowing registration of behavior feature quantities such as “object of curiosity/interest,” “line of sight,” “direction of body,” “facial expression,” and others, for example.

(Specific Example of this Service)

Data processing in the foregoing embodiment will be described next by referring to FIGS. 12-1 to 12-3 and by giving specific instances. FIGS. 12-1 to 12-3 show a specific example of this service and shows an example of data processing performed in a case in which a client (childcare worker) at an educational facility gives picture book reading to subjects (children).

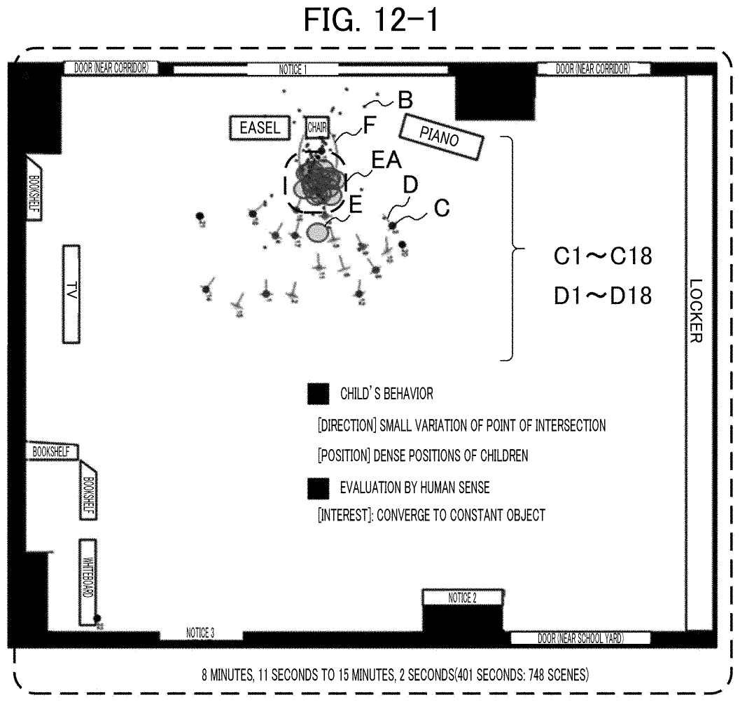

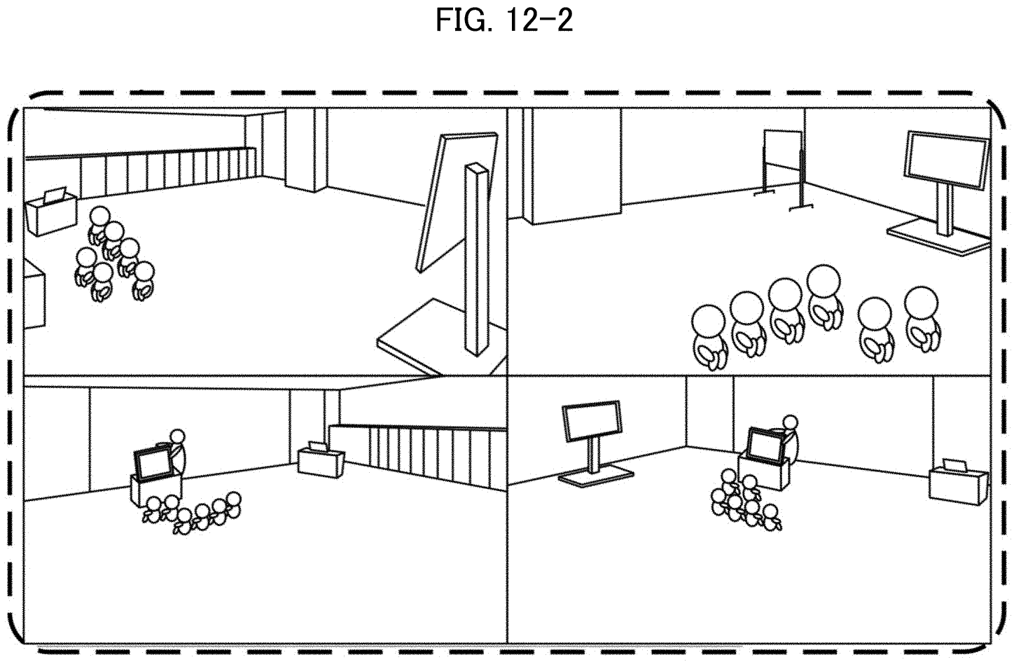

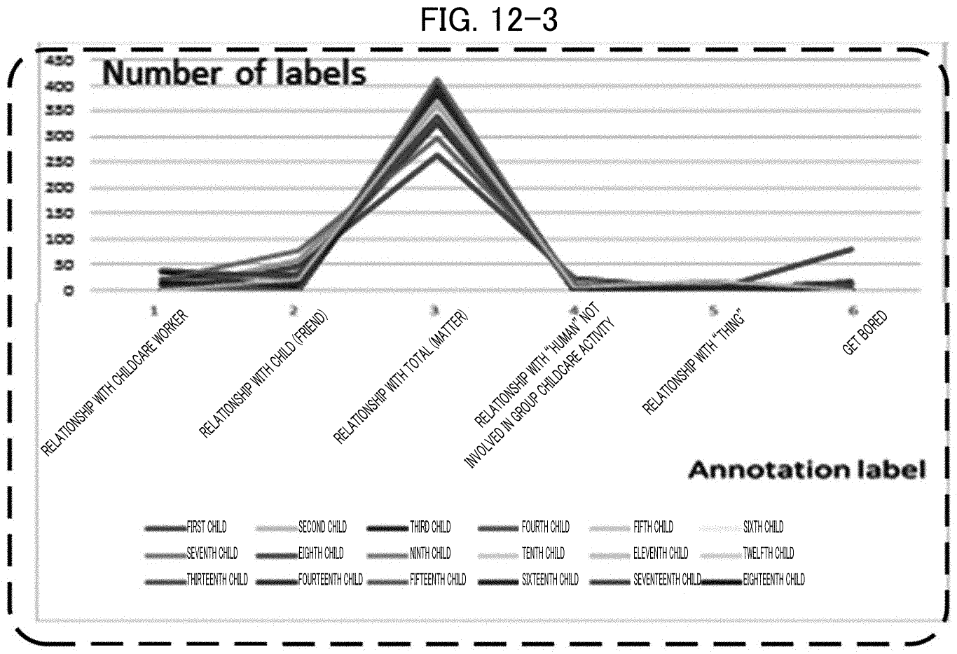

A plan view of the educational facility in which this service is used is drawn in FIGS. 12-1. The example of FIGS. 12-1 to 12-3 show information about a result of image processing including the positions or directions of lines of sight of 18 subjects (children) determined when the client (childcare worker) gives picture book reading to the 18 subjects (children). Namely, this example shows a plurality of points C (1 to 18) indicating the respective positions of the 18 subjects (children), and shows lines D (1 to 18) indicating the respective directions of the lines of sight of the subjects (children). A point B is a point of intersection of extension lines of a plurality of the lines D. A point without the line D indicates the client (childcare worker), for example, and indicates the position of a person other than a subject (child). In the example of FIGS. 12-1 to 12-3, frames E are present densely in a region EA, and a region F indicating variation of points B occupies a small area. Further, the points C converge to a periphery of a fixe object. Namely, in the example of FIGS. 12-1 to 12-3, the lines of sight of the 18 subjects (children) are pointed toward the client (childcare worker) giving the picture book reading and thus it is assumed that almost all the objects of interest of the children are the client (childcare worker) giving the picture book reading.

Captured images of the target educational facility taken from a plurality of angles are shown in FIG. 12-2. A graph showing the objects of interest of the 18 subjects (children) is shown in FIG. 12-3.

FIGS. 13-1 to 13-3 show a specific example of this service and shows an example in which the target educational facility is in free time.

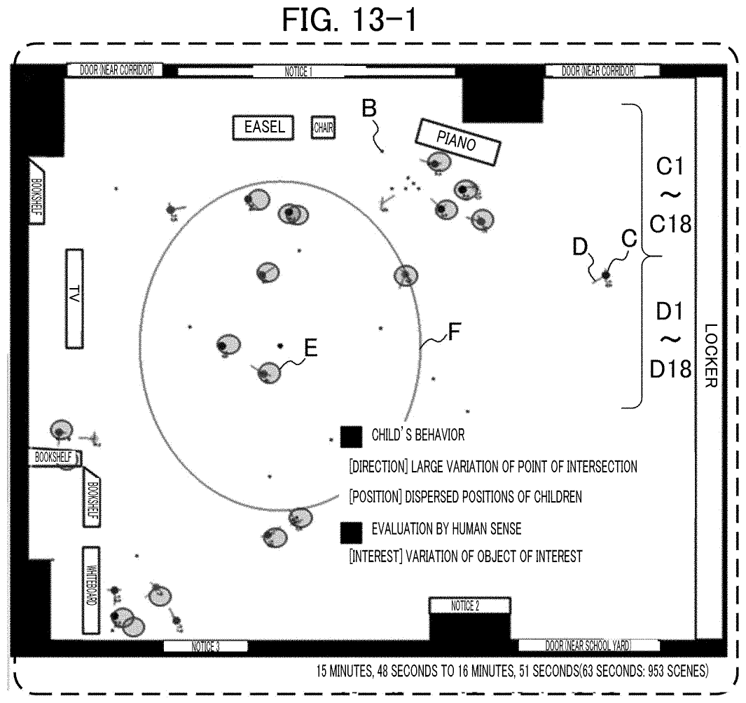

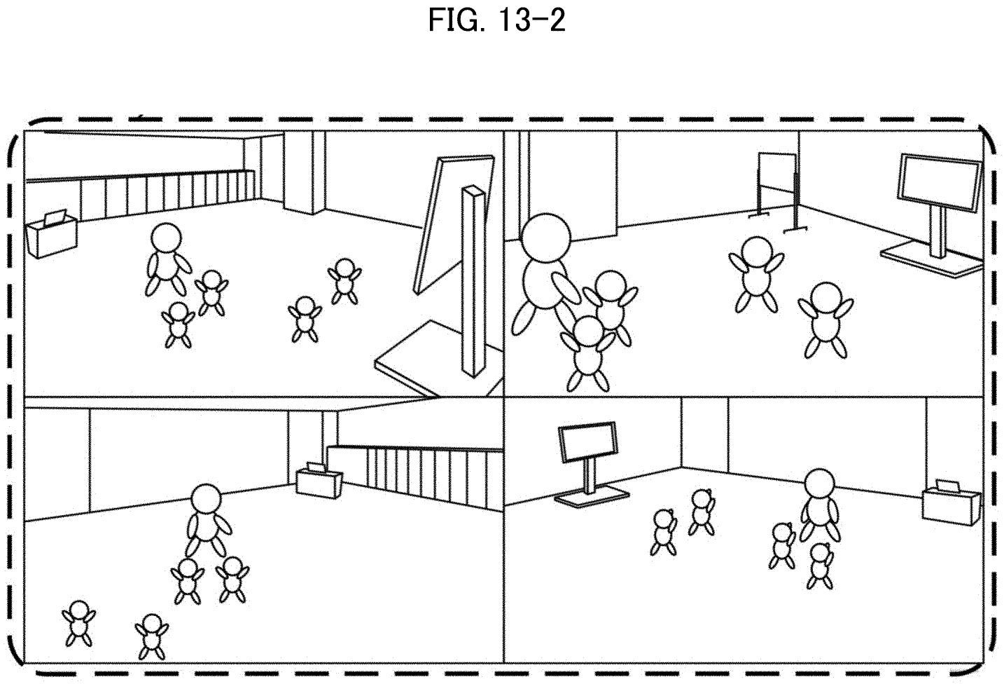

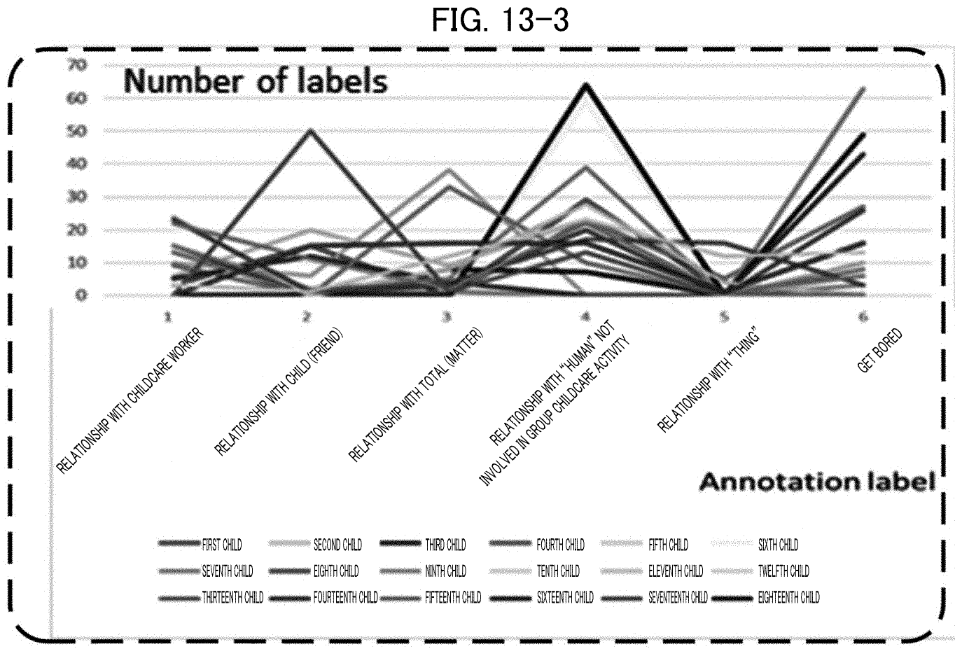

Like in FIG. 12-1, a plan view of the target educational facility, points C, lines D, points B, and frames E are displayed in FIG. 13-1. In the example of FIGS. 13-1 to 13-3, the points C indicating the respective positions of the 18 subjects (children) are dispersed and a region F indicating variation of the points B occupies a large area. The frames E are also dispersed. Namely, in the example of FIGS. 13-1 to 13-3, each of the 18 subjects (children) spends free time and these subjects point their lines of sight to different directions. Thus, it is assumed that there is variation in objects of interest.

Like in FIG. 12-2, captured images of the target educational facility taken from a plurality of angles are shown in FIG. 13-2. Like in FIG. 12-3, a graph showing the objects of interest of the 18 subjects (children) is shown in FIG. 12-3. Items listed as the respective objects of interest of the 18 subjects (children) include “relationship with childcare worker,” “relationship with child (fried),” “relationship with total (matter),” “relationship with “human” not involved in group childcare activity,” “relationship with “thing,” and “get bored.” This graph shows that there is variation in the objects of interest of the 18 subjects (children).

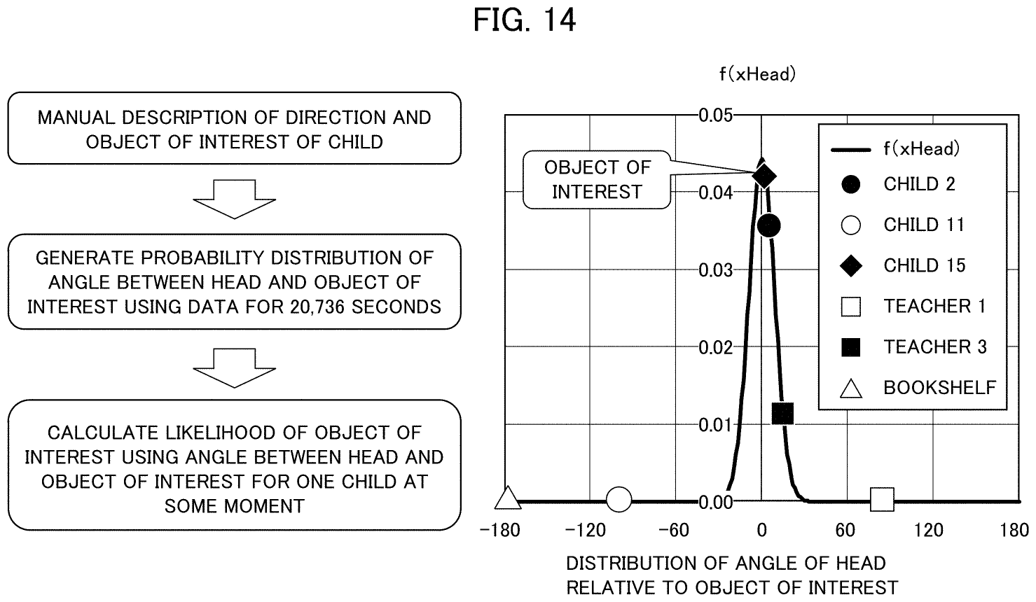

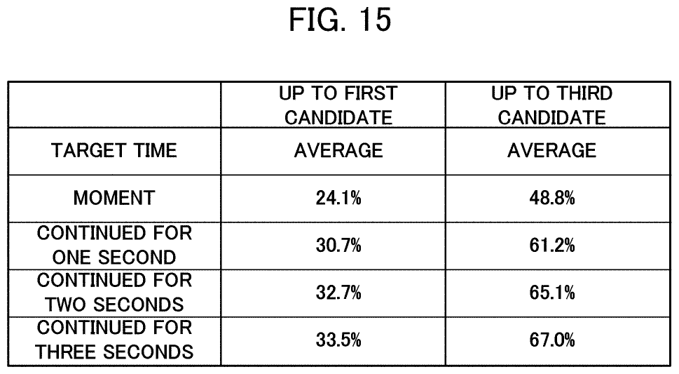

As described above, an object attracting the lines of sight of a subject (child) means that this object is an object of interest of this child. This will be described briefly and complementarily by referring to FIGS. 14 and 15.

FIG. 14 shows an example of a technique of calculating a likelihood distance to become a basis for supporting the principles of interest estimation.

As shown on the left side of FIG. 14, a likelihood distance can be calculated on the basis of an object of interest of a subject (child) recorded (described) by a client (childcare worker), and a probability distribution of an angle relative to an object of interest generated from a behavior observation result (a behavior observation result based on data about captured images lasting for 20,736 seconds, for example). While “angle” shown in FIG. 14 means “angle” on a two-dimensional plane, this is merely an example and “angle” may mean “angle” in three-dimensional space. As shown on the right side of FIG. 14, for example, the probability distribution of an angle relative to an object of interest can be expressed by a graph indicating a distribution of the angle of a head (f(xHead)) relative to the object of interest. Namely, the graph of FIG. 14 shows that, when the angle of the head of a subject (child) (f(xHead) at a certain moment is “0 (zero)” or an angle around zero, the object of interest exists at the destination of the line of sight of the subject (child).

FIG. 15 is a table showing a percentage of correct answers of interest estimation.

As described above, the graph of FIG. 14 shows a distribution of the angle of the head (f(xHead)) of a subject (child) relative to an object of interest at a certain moment. By contrast, as shown in the table of FIG. 15, from a viewpoint of “continuous time (one second or two seconds, for example)” not from a viewpoint of “moment,” a percentage of correct answers (reliability) of estimation of an object of interest changes. For example, an object of interest is estimated on the basis of the angle of the head of a subject (child) at a certain “moment,” and a percentage of correct answers (reliability) of an object of interest estimated as a first candidate is “24.1%” on average, as shown in FIG. 15. A percentage of correct answers (reliability) of objects of interest up to a third candidate is “48.8%” on average. An object of interest is estimated on condition that the angle of the head of the subject (child) is retained continuously for one second, and a percentage of correct answers (reliability) of an object of interest estimated as a first candidate is “30.7%” on average. A percentage of correct answers (reliability) of objects of interest up to a third candidate is “61.2%” on average. An object of interest is estimated on condition that the angle of the head of the subject (child) is retained continuously for two seconds, and a percentage of correct answers (reliability) of an object of interest estimated as a first candidate is “32.7%” on average to make increase by two points from the case of the retention for one second. A percentage of correct answers (reliability) of objects of interest up to a third candidate is “65.1%” on average to make increase by about four points from the case of the retention for one second. An object of interest is estimated on condition that the angle of the head of the subject (child) is retained continuously for three seconds, and a percentage of correct answers (reliability) of an object of interest estimated as a first candidate is “33.5%” on average to make increase by about one point from the case of the retention for two seconds. A percentage of correct answers (reliability) up to a third candidate is “67.0%” on average to make increase by about two points from the case of the retention for two seconds. As understood from the foregoing, when the angle of the head of a subject (child) is retained continuously at a prescribed angle for one or more seconds, a percentage of correct answers (reliability) is increased dramatically from the case of “moment.”

FIGS. 16-1 to 16-3 show an example of an analysis technique using a likelihood distance.

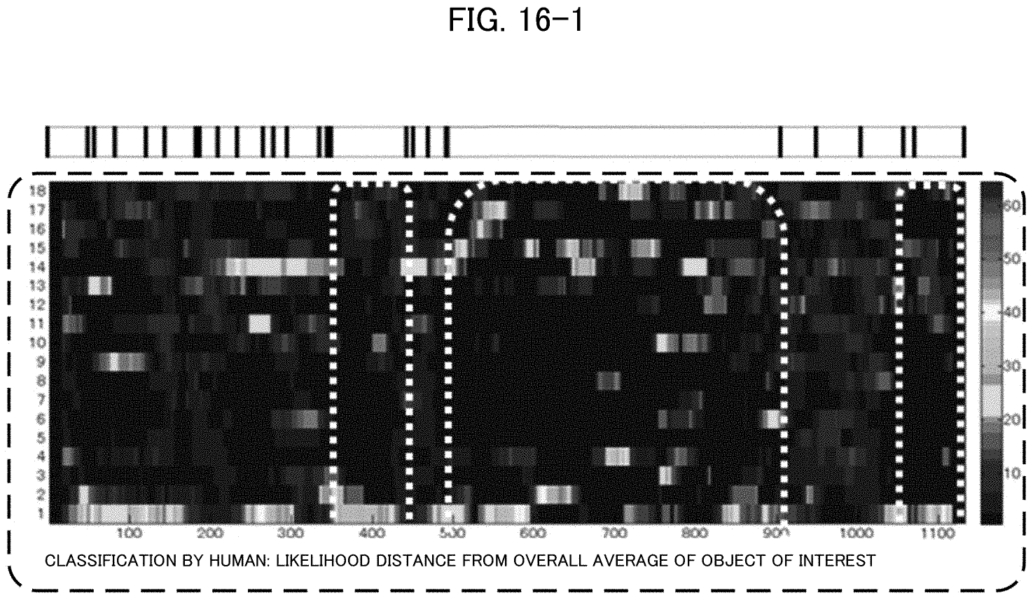

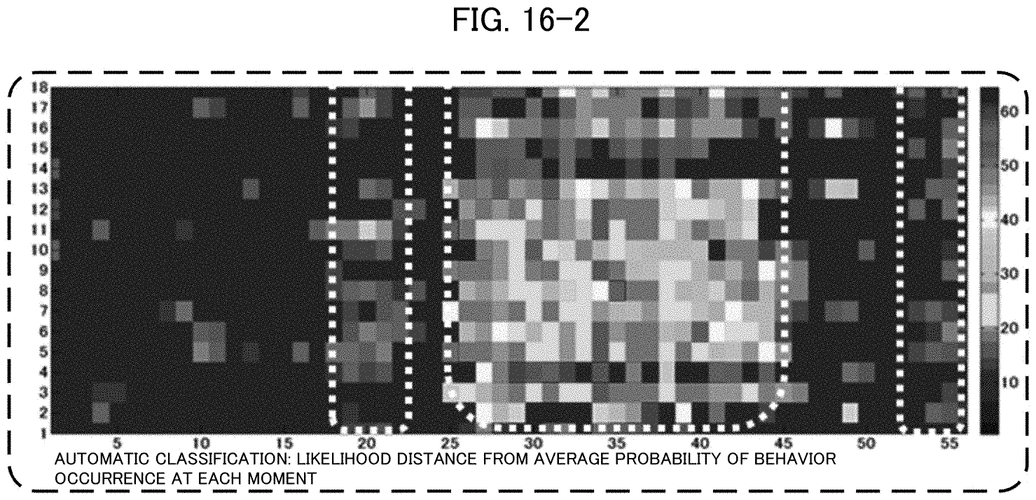

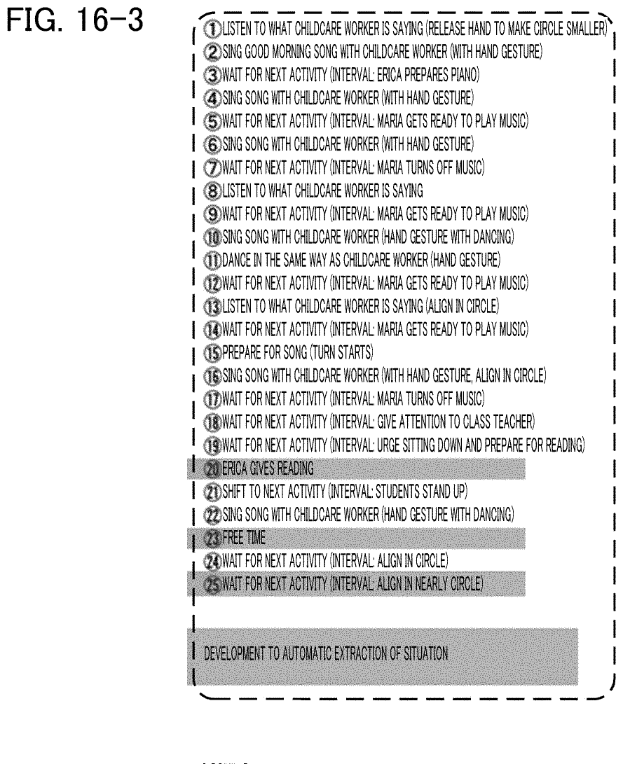

FIG. 16-1 indicated by dashes includes a graph showing a likelihood distance from an overall average of objects of interest of subjects (children) generated by “classification by human,” namely, generated by a person having expert skill such as a client (childcare worker). Further, FIG. 16-2 indicated by dashes includes a graph showing a likelihood distance from an average probability of behavior occurrences at each moment generated by “automatic classification,” namely, generated on the basis of a behavior observation result. In the example of FIGS. 16-1 to 16-3, a vertical axis shows subjects (children), and a horizontal axis shows extracted chronological change in each situation. In the example of FIGS. 16-1 to 16-3, contrast between white and black shows a distance of each subject (child) from an average of objects of interest.

As a specific example, it can be seen from an example corresponding to a first subject (child) in a lower part of FIG. 16-1 that, particularly in situations surrounded by white dots (20^{th} situation: “Erica gives reading,” 23^{rd} situation: “free time,” and 25^{th} situation: “wait for next activity”), this subject exhibits an object of interest (long likelihood distance) largely different from those of the other children. This tendency of the first subject (child) is also observed in “automatic classification” shown in FIG. 16-2. Namely, the example of FIGS. 16-1 to 16-3 indicate that “classification by human” classified by a person having expert skill and “automatic classification” classified by using various types of techniques relating to this service including the foregoing embodiment achieve comparable results in that the first subject (child) exhibits an object of interest different from those of the others, for example. In this way, the validity of “automatic classification” using the various types of techniques relating to this service is supported. In other words, these pieces of data suggest that an object of interest of a subject (child) can be estimated on the basis of the position and direction of the subject (child).

FIG. 16-2 indicated by dashes includes a graph showing a likelihood distance an average probability of behavior occurrences at each moment generated by “automatic classification,” namely, generated automatically on the basis of a behavior observation result. FIG. 16-3 indicated by dashes includes specific exemplary situations of a subject (child) classified automatically on the basis of a behavior observation result. More specifically, these situations include “listen to childcare worker's words,” “sing Good Morning Song with childcare worker,” “wait for next activity,” and “sing song with childcare worker.”

A result of the automatic classification achieved by this service can also be used as a basis for estimating the individual characteristic of a subject (child).

This allows a client (childcare worker), for example, to see a subject (child) or a group (class, for example) efficiently to which attention should be given from the client. In the presence of a subject (child) likely to get out of a group (class, for example), this subject may “get out” in various “styles” for various reasons. The automatic classification of this service allows the client (childcare worker) to see a behavior distribution responsive to a situation. By doing so, the client (childcare worker) becomes capable of determining a subject (child) or a group (class, for example) to which attention should be given on the basis of a likelihood distance of a behavior calculated from the behavior distribution. The client (childcare worker) also becomes capable of working cooperatively with a guardian of the subject (child) or the group (class, for example) to which attention should be given.

More specifically, the client (childcare worker) can see a friend relation of the subject (child) from a result of the estimation about the characteristic of the subject (child), and can estimate a level of caution needed for the subject (child) belonging to a group. The client (childcare worker) can also see the noisiness or concentration of the group (class, for example) in its entirety from results of the estimation about the characteristics of a plurality of subjects (children). In other words, the client (childcare worker) can quantify an air (atmosphere) in the group (class, for example) in its entirety and can make comparison with a different group. The client (childcare worker) can also form a population to which a plurality of subjects (children) leading a group (class, for example) is to belong. This achieves control over the quality of the group (class, for example).

This service achieves the following matters listed below. An object of interest of a subject (child) can be estimated on the basis of the momentum of the subject and a position where the subject stays. A subject (child) not giving attention to a client (childcare worker) or a subject (child) giving too much attention to a client (childcare worker) can be estimated. Physical or psychological closeness between subjects (children) or between a subject (child) and a teacher can be estimated. A level of participation of a subject in activity can be estimated by encouraging synchronization between subjects (children) or between a subject (child) and a teacher. Combining classification of a subject (child) and classification of a group behavior achieves pluralistic grasp of a character from viewpoints including activity to which the subject (child) is devoted or not devoted, and a target the subject (child) is good at or not good at.

Exemplary applications of the information processing system according to the embodiment of the present invention will be described briefly. Like in the foregoing embodiment, in a situation where this service is used in childcare and educational fields, the information processing system is expected to be applied to “early detection of child developmental disability (longitudinal development research),” “estimation of appetite for learning (verification of educational effect, for example),” “estimation of risk factor in educational activity (prevention of accident or bullying, for example),” “estimation of proper vocational task (estimation of educational suitability, for example),” and others. These exemplary applications can be implemented at “childcare or educational facility,” “education research institution,” “education administrative institution,” and others.

In the example described in the foregoing embodiment, this service is used in childcare and educational fields. However, this service is further usable in other fields. More specifically, this service is usable in medical and caregiving fields, marketing fields, or robot service fields. If this service is used in medical and caregiving fields, for example, it is expected to be applied to “estimation and judgment of dementia (early detection and estimation of development of dementia),” “estimation of level of mental health (stress management of patient or staff),” and others. These exemplary applications can be implemented at “medical facility,” “caregiving facility,” “company human resource department,” and others, for example. Likewise, in a situation where this service is used in marketing fields, for example, it is expected to be applied to “evaluation of psychological state of customer (extraction of customer's need),” “presentation of design reflecting interest (estimation of optimization for facility arrangement, for example),” and others. These exemplary applications can be implemented at “commercial facility,” “design business,” and others, for example. Likewise, in a situation where this service is used in robot service fields, for example, it is expected to be applied to “estimation of human feeling using domestic robot, caregiving robot, or work-site robot,” namely, expected to be applied to customer service, livelihood support, stress relaxation, and others using a robot. These exemplary applications can be implemented at companies developing robots or at sites where robots are used (by individuals or legal persons, for example).

While the embodiment has been described by employing a method of classification into the sixth types (first classification to sixth classification), for example, this is not the only case. More specifically, the server 1 may employ any method other than the foregoing method of classification into the six types of “fields.” For example, classification may be classified into eight types of “fields,” or the concepts of the foregoing six types of “fields” may be changed, if appropriate. In the foregoing embodiment, the six types are largely defined by determining “fields” of the first classification to the third classification to be “field” types each formed by a group, and by determining “fields” of the fourth classification to the sixth classification to be “field” types each formed by a specific individual. However, this is not the only case. Namely, the server 1 can classifies “field” using a “field” type defined using any other concepts.

In the foregoing embodiment, further, “one unit” in Claims has been described as “situation.” However, this is not the only case. The server 1 may use any concept including the foregoing “situation” as “one unit.” For example, the server 1 may use a concept of “short time of about one second” as “one unit.” More specifically, according to a technique adoptable by the server 1, an object of interest of an individual is classified at short intervals of one about one second, and resultant classifications are combined to estimate an object of interest of a group at intervals of about one second or at slightly longer intervals of about 10 seconds. Then, “field” formed by a group estimated therefrom is determined, and a point of change between the fields is used for separating captured images taken continuously into “one unit.” Namely, “one unit” in Claims does not always have a range of a constant period and is not always fixed but it means an object of interest of a group and eventually means a duration when activity continues in the group. A point of change in the activity may be used for defining “one unit.”

In the foregoing embodiment, the program installed on the server 1 is executed to perform the characteristic information estimation process shown in FIG. 5, thereby realizing this service. However, the program to be executed for realizing this service may not be a program installed on the server 1 but may be a program installed on the client terminal 2, for example.

In the foregoing embodiment, respective behavior observation results about a plurality of subjects are acquired by image analysis processing on the basis of data about captured images (moving image data). However, this is not the only method of acquiring a behavior observation result. For example, a sensor such as a visual sensor (camera) or an acceleration sensor may be attached directly to each subject, and a behavior observation result about each subject may be acquired on the basis of detection information from the sensor attached to each subject.

A technique of calculating a likelihood distance is not limited to the one described in the foregoing embodiment. For example, a Topic (topic) model can be used in automatic classification of a situation and calculation of a likelihood distance of a subject (child).

“Topic model” mentioned herein is a model based on the assumption that a document is generated on the basis of a plurality of latent Topics. In the Topic model, each word forming the document is assumed to appear according to a probability distribution belonging to a prescribed Topic. Namely, in the Topic model, a distribution of an appearance frequency of a word forming the document is estimated to allow analysis of similarity between Topics and that meaning. This Topic model is used in automatic classification of a situation and calculation of a likelihood distance according to this service. As a specific example, if a group to which a subject (child) belongs is “English class,” association of each behavior of the subject (child) is first established with “English song,” “interest in human,” and “interest in thing” distributed as Topics. Next, on the basis of the position and speed of each subject (child) obtained from a behavior observation result, a behavior of each subject (child) is classified. A specific technique of classifying a behavior on the basis of the position and speed of each subject (child) is not particularly limited. For example, a technique employing clustering without teacher using a Hierarchical Dirichlet Process Hidden Markov Model (HDP-HMM) may be used. Then, a likelihood distance of a behavior of a subject (child) can be calculated on the basis of a behavior probability distribution in each class employing clustering without teacher using Latent Dirichlet Allocation (LDA), for example.

In the foregoing embodiment, only a depression angle is shown as an example of “direction” of a subject (child). However, this is not the only example but the angle may include an elevation angle. Namely, the motion of the subject (child) is not limited to tilting of a head or a body to the right and left in the horizontal direction but it may also include tilting up and down in the vertical direction.

In the foregoing embodiment, the configuration of the information processing system of the present invention includes the server 1 and the client terminal 2. However, this is merely an exemplary configuration for fulfilling the purpose of the present invention and is not the particularly limiting configuration. More specifically, while only one client terminal 2 is shown in the example of FIG. 1 for the convenience of description, the number of the client terminals 2 can be set appropriately in response to the number of clients to receive presentation of this service. In the foregoing embodiment, data about captured images is transmitted through the client terminal 2 to the server 1 via the prescribed network N. Alternatively, the data about captured images may be input from the imaging device 3 directly to the server 1 by wire. In the foregoing embodiment, the client terminal 2 and the imaging device 3 are provided separately. Alternatively, the client terminal 2 may include an imaging unit (video camera unit, for example) (the client terminal 2 and the imaging unit 3 may be configured integrally). In the foregoing embodiment, further, one imaging device 3 is connected to one client terminal 2. However, this is not the only case. In this service, a plurality of the imaging devices 3 may be connected to one client terminal 2, or one imaging device 3 may be connected to a plurality of the client terminals 2. In such cases, the client terminal 2 becomes capable of receiving images (moving images) simultaneously from a plurality of imaging devices, for example.

Each hardware structure in FIG. 3 is shown merely as an example for fulfilling the purpose of the present invention and is not the particularly limiting structure.

The functional block diagram in FIG. 4 is shown merely as an example and is not the particularly limiting diagram. Namely, as long as the information processing system has a function allowing implementation of the foregoing series of processes entirely, functional blocks for realizing this function are not particularly limited to the examples shown in FIG. 4. Namely, the characteristic information estimation process shown in FIG. 5 may be performed by a single information processing device (server 1, for example), or by an information processing system including a plurality of information processing devices (server 1 and client terminal 2). The characteristic information estimation process shown in FIG. 5 may be performed as a part of an application program installed on the client terminal 2.

The locations of the functional blocks are not limited to those in FIG. 4 but may be determined arbitrarily. For example, at least some of the functional blocks of the server 1 may be provided to the client terminal 2, or vice versa. One functional block may be configured using a hardware unit, or may be configured using a combination with a software unit.

To realize the process of each functional block by software, a program configuring the software is installed from a network or a storage medium on a computer, for example. The computer may be a computer incorporated into dedicated hardware. The computer may be a computer to become available for use for fulfilling various types of functions by installing various types of programs such as a server, and additionally, a general-purpose smartphone or personal computer, for example.

The storage medium including the foregoing program is configured not always using a removable medium distributed separately from a device body for providing the program to each client but is also configured using a storage medium provided to each client in a state of being incorporated in advance into the device body, for example.

In this description, steps describing the program stored in the storage medium certainly include processes to be performed in chronological order according to the order of the steps, and further include processes not to necessarily be performed in chronological order but to be performed in parallel or individually.

As another way of stating the foregoing, the information processing device to which the present application is applied can be embodied in a wide variety of ways having the configurations as follows:

The information processing device to which the present invention is applied is required only to include:
acquisition means (observation result acquisition unit 113 in FIG. 4, for example), using a prescribed location in a prescribed time period as one unit, the acquisition means extracting a behavior observation result about a person on the basis of each of a plurality of the units for each of a plurality of persons;
object-of-interest estimation means (object-of-interest estimation unit 114 in FIG. 4, for example), on the basis of the behavior observation result acquired for each of a plurality of the persons and each of a plurality of the units, the object-of-interest estimation means estimating an object of interest of an individual; and
field classification means (field classification unit 115 in FIG. 4, for example), on the basis of a result of the estimation by the object-of-interest estimation means obtained for each of a plurality of the persons and each of a plurality of the units, the field classification means classifying each of a plurality of the units into one or more types of fields from among one or more types of fields formed by a group and one or more types of fields formed by a specific individual. This makes it possible to properly estimate a situation in which an object of interest of a subject (person) has appeared (whether this situation is a situation formed by a group or a situation formed by a specific individual).

The object-of-interest estimation means can estimate an object of interest of a prescribed person in a prescribed unit from one or more elements from among three elements including a human, a thing, and a matter. This makes it possible to estimate the type of an object of interest of a subject (person).

Psychological trait estimation means (psychological trait estimation unit 116 in FIG. 4, for example) can be provided further. When the object-of-interest estimation means estimates an object of interest of the prescribed person in the prescribed unit, the psychological trait estimation means estimates the psychological trait of the prescribed person on the basis of a difference between a behavior observation result about the prescribed person and an average of behavior observation results about other persons in the prescribed unit. This makes it possible to properly estimate the psychological trait of a subject (person).

The psychological trait can include six factors of openness, positiveness, diligence, stability, adaptability, and leadership. This makes it possible to analyze the psychological trait of a subject (person) from many sides.

Developmental characteristic estimation means (developmental characteristic estimation unit 117 in FIG. 4, for example) can be provided further. On the basis of a difference between a feature of a long-term transition of the psychological trait of the prescribed person estimated by the psychological trait estimation means and a feature of a long-term transition of a standard psychological trait, the developmental characteristic estimation means estimates the developmental characteristic of the prescribed person. This makes it possible to properly estimate the developmental characteristic of a subject (person).

The acquisition means can acquire a behavior observation result about a person on the basis of each of a plurality of the units for each of a plurality of the persons. The behavior observation result contains information about at least one of a position and a direction. This makes it possible to properly estimate a situation in which an object of interest of a subject (person) has appeared (whether this situation is a situation formed by a group or a situation formed by a specific individual) on the basis of the position or direction, or on the basis of the position and direction of the subject (person).

The present invention is further applicable to an information processing method or a program.

### EXPLANATION OF REFERENCE NUMERALS

1 . . . Server, 2 . . . Client terminal, 3 . . . Imaging device, 11 . . . CPU, 21 . . . CPU, 24 . . . Display unit, 111 . . . Captured image acquisition unit, 112 . . . Captured image analysis unit, 113 . . . Observation result acquisition unit, 114 . . . Object-of-interest estimation unit, 115 . . . Field classification unit, 116 . . . Psychological trait estimation unit, 117 . . . Developmental characteristic estimation unit, 118 . . . Output data generation unit, 119 . . . Information transmission unit, 211 . . . Captured image management unit, 212 . . . Output information acquisition unit, 500 . . . Classification DB, 600 . . . Psychological trait DB, 12-1 to 12-3, 13-1 to 13-3, 16-1 to 16-3 . . . Region, B . . . Point of intersection of lines indicating directions of subjects, C . . . Point indicating position of subject, D . . . Line indicating direction of subject, E . . . Frame indicating object of interest of subject, EA . . . Region, F . . . Region indicating variation of point of intersection of lines indicating directions of subjects

1. An information processing device comprising: acquisition means, using a prescribed location in a prescribed time period as one unit, the acquisition means extracting a behavior observation result about a person on the basis of each of a plurality of the units for each of a plurality of persons;
object-of-interest estimation means, on the basis of the behavior observation result acquired for each of a plurality of the persons and each of a plurality of the units, the object-of-interest estimation means estimating an object of interest of an individual; and
field classification means, on the basis of a result of the estimation by the object-of-interest estimation means obtained for each of a plurality of the persons and each of a plurality of the units, the field classification means classifying each of a plurality of the units into one or more types of fields from among one or more types of fields formed by a group and one or more types of fields formed by a specific individual.

2. The information processing device according to claim 1, wherein the object-of-interest estimation means estimates an object of interest of a prescribed person in a prescribed unit from one or more elements from among three elements including a human, a thing, and a matter.

3. The information processing device according to claim 2, further comprising psychological trait estimation means, when the object-of-interest estimation means estimates an object of interest of the prescribed person in the prescribed unit, the psychological trait estimation means estimating the psychological trait of the prescribed person on the basis of a difference between a behavior observation result about the prescribed person and an average of behavior observation results about other persons in the prescribed unit.

4. The information processing device according to claim 3, wherein the psychological trait includes six factors of openness, positiveness, diligence, stability, adaptability, and leadership.

5. The information processing device according to claim 4, further comprising developmental characteristic estimation means, on the basis of a difference between a feature of a long-term transition of the psychological trait of the prescribed person estimated by the psychological trait estimation means and a feature of a long-term transition of a standard psychological trait, the developmental characteristic estimation means estimating the developmental characteristic of the prescribed person.

6. The information processing device according to claim 1, wherein
the acquisition means acquires a behavior observation result about a person on the basis of each of a plurality of the units for each of a plurality of the persons, the behavior observation result containing information about at least one of a position and a direction.

7. An information processing method executed by an information processing device, comprising:
an acquisition step, using a prescribed location in a prescribed time period as one unit, the acquisition step extracting a behavior observation result about a person on the basis of each of a plurality of the units for each of a plurality of persons;
an object-of-interest estimation step, on the basis of the behavior observation result acquired for each of a plurality of the persons and each of a plurality of the units, the object-of-interest estimation step estimating an object of interest of an individual; and
a field classification step, on the basis of a result of the estimation by the object-of-interest estimation means obtained for each of a plurality of the persons and each of a plurality of the units, the field classification step classifying each of a plurality of the units into one or more types of fields from among one or more types of fields formed by a group and one or more types of fields formed by a specific individual.

8. A non-transitory computer-readable storing medium that stores a program for causing a computer for controlling an information processing device to perform a control process comprising:
an acquisition step, using a prescribed location in a prescribed time period as one unit, the acquisition step extracting a behavior observation result about a person on the basis of each of a plurality of the units for each of a plurality of persons;
an object-of-interest estimation step, on the basis of the behavior observation result acquired for each of a plurality of the persons and each of a plurality of the units, the object-of-interest estimation step estimating an object of interest of an individual; and
a field classification step, on the basis of a result of the estimation by the object-of-interest estimation means obtained for each of a plurality of the persons and each of a plurality of the units, the field classification step classifying each of a plurality of the units into one or more types of fields from among one or more types of fields formed by a group and one or more types of fields formed by a specific individual.
